(19) United States
(12) Patent Application Publication
(10) Pub. No.: US 2016/0228673 A1
(43) Pub. Date: Aug. 11, 2016

(54) HEAD BONE CONDUCTION DEVICE AND METHOD

(71) Applicant: Kacheung CHOW, Hong Kong (CN)

(72) Inventor: Kacheung CHOW, Hong Kong (CN)

(21) Appl. No.: 15/025,136

(22) PCT Filed: Jan. 21, 2015
(86) PCT No.: PCT/CN2015/071182

§ 371 Date: Mar. 25, 2016

(30) Foreign Application Priority Data
Feb. 14, 2014 (CN) 201410052071.6

(51) Int. Cl. A61M 21/00; A61B 5/04; A61H 23/00; A61B 5/048

(52) U.S. Cl. CPC A61M 21/00; A61B 5/048; A61B 5/04014; A61H 23/004; A61M 2021/0022; A61M 2021/0027; A61M 2205/3375; A61H 2205/02; A61H 2201/1607

(57) ABSTRACT

The present disclosure relates to a head bone conduction device and method. The head bone conduction device includes: a head fixing unit, a brain wave detection connecting ends, a frequency signal generating circuit, an audio device and an audio mixer. Seven independent bone conduction units are adopted for operation, and at least one bone conduction unit is adopted for operation and is arranged at a suitable position of a head. Audio information received by the bone conduction unit is converted into mechanical vibration, thus vibration force, vibration sensation and mechanical vibration are generated simultaneously on a region of a cranial cortex. Accompanying with the ear bone mechanical vibration, a user will feel a mixed sensation including auditory and positional sense, or namely ‘third auditory’. The frequency signal generation circuit generates specific frequency signals according to detected brain wave signals to the audio mixer. The frequency signals will then be mixed with regular audio signal, whose change would be aware by the user for secondary confirmation.

### TECHNICAL FIELD

The present disclosure relates to bone conduction device technology, and more particularly, to a head bone conduction device and a method.

### BACKGROUND

Brain functions are required to be called in an effective way to save and/or access information in a brain. The performance of brain functions, such as information access, logical thinking and memorization, connects closely and directly with the network building of cortical neurons which acts as the only basic unit for information transfer and memory storage. As such, an efficient application of brain functions would help people to focus on learning, which will help memorize greatly.

When the cerebral cortex is activated or its' active status changes, frequency and spectrum of brain wave would be induced to change. It would be much easier for people to learn when a specific brain wave spectrum presents which indicates a high concentration of attention.

Alpha brain waves (a brain wave) generally occur in childhood. When people grown up, his brain would be dominated by beta brain wave (β brain wave) to perceive and conceive outside information. The existence of beta brain wave indicates a tendency of left brained mode while the existence of alpha brain wave indicates a tendency of right brained mode. Left brained mode is advantageous on logical thinking and comparison between different situations to judge right from wrong. On the other hand, right brained mode is advantageous on fast learning, memorize, visual and audio skills. As such, it is required to understand how to switch the operation mode of a brain to utilize the individual advantage of right brained mode when alpha brain wave occurs.

The brain wave spectrums can be classified as follows:

1. spectrum 4-7 Hz, which can be defined as sleeping status;

2. spectrum 7-12 Hz, which can be defined as a super learning status with alpha brain waves present. Generally, people are more focused and more efficient to learn under this status; and

3. spectrum 13-30 Hz, which can be defined as a logical thinking status with beta brain waves present. Generally, people feel more nervous and more likely tired due to a high consumption of energy.

The existing bone conduction devices for learning basically include a binaural headphone or a single-ear headphone as a core component operated together with a bone conduction unit. Alternatively, some existing bone conduction devices further include brain wave detection unit to monitor the change of brain wave when a brain is stimulated by visual and/or audio stimulation, which can help an external computer to regulate the brain waves.

However, the existing bone conduction devices for learning are complicated for user to operate. Moreover, the existing bone conduction devices are susceptible to interference from headphones and other components.

### SUMMARY

The objective of the present disclosure is to provide a head bone conduction device and a method, in order to overcome the problems that the existing bone conduction device for learning are complicated for user to operate and susceptible to interference from headphones and other components. Moreover, it is difficult for a user to identify his learning status with the existing bone conduction device for learning.

The present invention discloses a head bone conduction device, including: a head fixing unit to support and secure the device to the head of a user; at least one bone conduction unit which is configured to convert an audio information from a sound mixer into mechanical vibration, and conduct the mechanical vibration to a region of cranial cortex which is attached to; at least one brain wave detection end which is configured to detect brain wave signal of the cranial cortex; at least a frequency signal generating circuit to generate the frequency signal within audible frequency based on the detected brain wave signal from the brain wave detection end; a first audio apparatus to provide a first audio signal; a sound mixer to receive at least one frequency signal within audible frequency, and mix the frequency signal with a first audio signal to obtain audio information.

According to the head bone conduction device in an embodiment of the present disclosure, wherein the head fixing unit further includes a head support, and at least one brain wave detection end which is located inner side of the head support of the head fixing unit.

According to the head bone conduction device in an embodiment of the present disclosure, wherein the head fixing unit is of a “U” shaped frame structure and comprises a head support at an upper portion and two opposite clamping portions at side portions.

According to the head bone conduction device in an embodiment of the present disclosure, wherein the device further includes at least one elongated contact arm, one end of each elongated contact arm movably is connected to the head fixing unit while another end connected to the bone conduction unit.

According to the head bone conduction device in an embodiment of the present disclosure, wherein the device further comprises a head fixing unit to support and secure the device to the head of a user, the head fixing unit comprises: a head support; a plurality of elongated contact arms which are located around the head support, one end of each elongated contact arms is connected to the head fixing unit, while another end of each elongated contact arms is connected to the brain wave detection end and/or the bone conduction unit; and, the plurality of elongated contact arms can be clamped to the head of a user to attach the one brain wave detection end and the bone conduction unit to a scalp of the cranial cortex.

According to the head bone conduction device in an embodiment of the present disclosure, wherein the device comprises seven bone conduction units, and the seven bone conduction units are connected to the head support respectively via seven elongated contact arms to form a helmet shape.

According to the head bone conduction device in an embodiment of the present disclosure, wherein the device further comprises at least one switch, each switch is correspondingly connected to the bone conduction unit.

According to the head bone conduction device in an embodiment of the present disclosure, wherein the device further comprises at least one elongated switch arm which is connected to the corresponding bone conduction unit to conduct mechanical vibration, each elongated switch arm is provided with an individual switch to control the corresponding bone conduction unit.

According to the head bone conduction device in an embodiment of the present disclosure, wherein the device further comprises a micro unit which is connected to the switch to control on-off between the sound mixer and the bone conduction unit according to the switch of the elongated switch arm.

According to the head bone conduction device in an embodiment of the present disclosure, wherein the frequency signal generating circuit further comprises: a plurality of oscillators which are connected in parallel to generate frequency signals within audible frequency; a frequency selector which is connected to a plurality of oscillators in series to conduct one of oscillators when the detected brain wave signal is an alpha brain wave; and a timer which is connected to the plurality of oscillators in series and configured to control a delaying period to conduct the plurality of oscillators.

According to the head bone conduction device in an embodiment of the present disclosure, wherein the plurality of oscillators further comprises a 200 Hz oscillator, a 400 Hz oscillator and a 600 Hz oscillator; and the delaying period by the timer is 0 second, 0.5 second, 1 second or 1.5 seconds.

According to the head bone conduction device in an embodiment of the present disclosure, wherein the bone conduction device further includes a front stage amplifier which is connected to a brain wave detection end to amplify the brain wave signal detected by the brain wave detection end for a first time; a frequency filter which is connected to the front stage amplifier to filter the amplified brain wave signal from the front stage amplifier; a post stage amplifier which is connected to the frequency filter to amplify the filtered brain wave signal from the frequency filter for a second time and compensate its' attenuation and loss in amplitude; and a low pass filter which is connected to the post stage amplifier to receive the second amplified brain wave signal from the post stage amplifier and to allow a brain wave signal of specific frequency to pass through; and a brain wave signal output end which is connected to the low pass filter to receive the brain wave signal filtered by the low pass filter.

According to the head bone conduction device in an embodiment of the present disclosure, wherein the device further comprises an indicating light to be triggered when the detected brain wave signal from the brain wave detection end is alpha brain wave.

According to the head bone conduction device in an embodiment of the present disclosure, wherein the frequency signal generating circuit is further configured to feedback the detected brain wave signal from the brain wave detection end to the sound mixer; and the sound mixer mix the detected brain wave signal with a regular audio signal to obtain an audio information, and send the audio information to at least one bone conduction unit.

According to the head bone conduction device in an embodiment of the present disclosure, wherein the device comprises more than one bone conduction units and each bone conduction units conducts its mechanical vibration in sequence or in random.

According to the head bone conduction device in an embodiment of the present disclosure, wherein the bone conduction unit comprises a phalanx conduction portion which is located backward the cranial cortex to conduct the mechanical vibration from the bone conduction unit to a phalanx.

According to the bone conduction device in an embodiment of the present disclosure, wherein the brain wave signal is an α brain wave signal.

According to the head bone conduction device in an embodiment of the present disclosure, wherein a manual switch is connected between the frequency signal generating circuit and the sound mixer.

According to the head bone conduction device in an embodiment of the present disclosure, wherein the device further comprises at least one audio amplifier whose quantity is in correspondence with the number of the bone conduction unit, and the audio amplifier is connected between the sound mixer and the bone conduction unit.

According to the head bone conduction device in an embodiment of the present disclosure, wherein the device further comprises a second audio apparatus to provide a second audio signal to the sound mixer when the brain wave signal detected by the brain wave detection end is an α brain wave.

According to the head bone conduction device in an embodiment of the present disclosure, wherein both the first audio apparatus and the second audio apparatus are audio input terminal.

The present disclosure also provides a bone conduction method, including: attaching at least one bone conduction unit to a region of a cranial cortex to conduct mechanical vibration; detecting a brain wave signal of the cranial cortex by the brain wave detection end; generating at least one frequency signal based on the brain wave signal detected by the brain wave detection end; and transforming a mixed signal into mechanical vibration, wherein the mixed signal comes from a regular audio signal mixed with the frequency signal.

According to the head bone conduction method in an embodiment of the present disclosure, wherein generating at least one frequency signal based on the brain wave signal detected by the brain wave detection end further comprises: judging whether the detected brain wave signal is an alpha brain wave or not; if the detected brain wave signal is an alpha brain wave signal, classifying the detected brain wave according to different frequency; and generating the frequency signal based on the detected brain wave.

According to the head bone conduction method in an embodiment of the present disclosure, wherein the method further comprises: adjusting a triggering time for the frequency signal by a timer.

According to the head bone conduction method in an embodiment of the present disclosure, wherein the method further comprises: Dividing the cranial cortex into seven regions, wherein three regions are located in the left side, another three regions are located in the right side and a region is located at the center; When the brain wave signal is an alpha brain wave, a bone conduction unit gets attached to different regions to identify which region the alpha brain wave comes from.

According to the head bone conduction method in an embodiment of the present disclosure, wherein seven bone conduction units individually get attached to seven regions of cranial cortex to conduct mechanical vibration in random or in sequence.

According to the head bone conduction method in an embodiment of the present disclosure, wherein the steps of detecting a brain wave signal of the cranial cortex by the brain wave detection end and generating at least one frequency signal based on the brain wave signal detected by the brain wave detection end further comprises: judging whether the detected brain wave signal is an α brain wave or not, if the detected brain wave signal is an alpha brain wave signal, transforming a mixed signal into mechanical vibration wherein the mixed signal comes from a regular audio signal mixed with the alpha brain wave signal.

According to the head bone conduction method in an embodiment of the present disclosure, wherein the method further includes: pressing the bone conduction unit by finger to conduct mechanical vibration from the bone conduction unit to phalanx to enable a user to identify the specific position where a combination of mechanical vibration sense from cranial cortex and from finger acting on.

According to the head bone conduction method in an embodiment of the present disclosure, wherein the method further includes: if the detected brain wave signal is an alpha brain wave signal, a second audio signal is inputted from the second audio apparatus and be mixed with the frequency signal.

As such, in the present disclosure, bone conduction units are individually attached to the cranial cortex to conduct mechanical vibration. Meantime, attention is drawn to the region where mechanical vibration acting on to awake the cranial cortex. After such a period of action, the brain would turn to a status of receiving mechanical vibration. By combining the brain wave signal from the cranial cortex and audio signal from an external audio device, a user would judge his own status when he realizes the change of mechanical vibration. Attention is kept drawn to the regions where mechanical vibration acting on the cranial cortex such that the attention would become long-lasting, which means enhancing mental focus. Moreover, the present invention helps the brain functioning and prevents brain problems such as memory loss. Further, comparing with the existing head bone conduction device, the present invention is simpler in structure and not susceptible to interference from other components such as headphones.

### BRIEF DESCRIPTION OF THE DRAWINGS

FIG. 1 is a schematic diagram of an embodiment of the present invention;

FIG. 2 is a partition view of a cranial cortex in an embodiment of the present invention;

FIG. 3 is a schematic diagram of an embodiment of the present invention attached to a region of cranial cortex;

FIG. 4 is a structural schematic diagram of another embodiment of the present invention;

FIG. 5 is a bottom view of FIG. 4;

FIG. 6 is a schematic diagram of an application of the present invention with seven bone conduction units onto cranial cortex;

FIG. 7 is a circuit schematic of the present invention;

FIG. 8 is another circuit schematic of the present invention; and

FIG. 9 is a circuit schematic of the control circuit of the present invention having seven bone conduction units.

### DETAILED DESCRIPTION

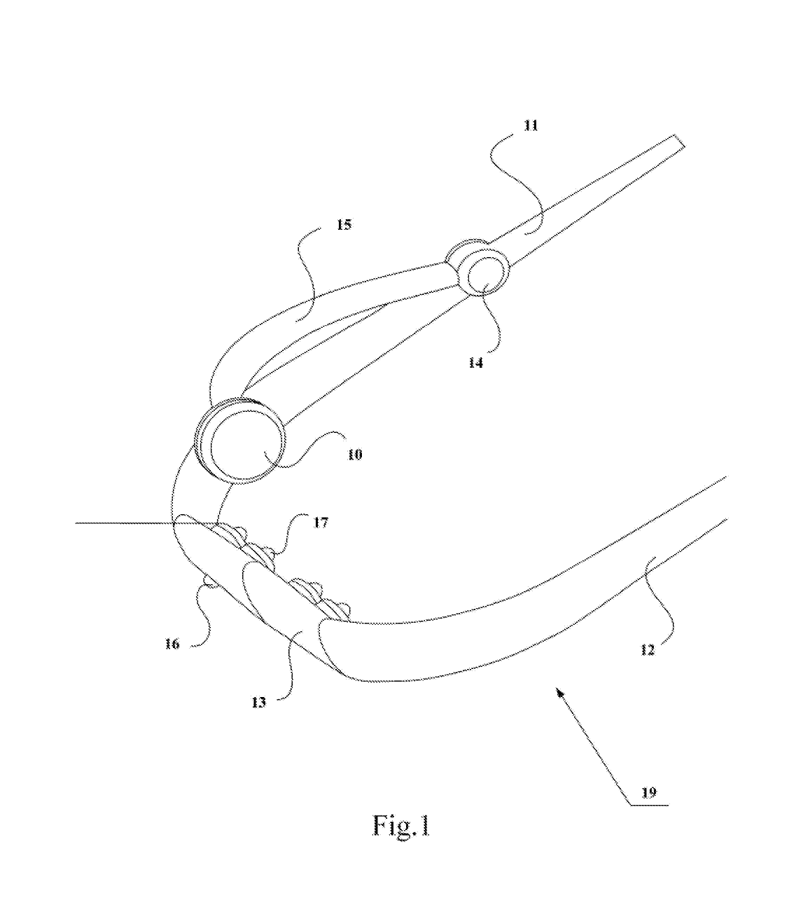

FIG. 1 is a schematic diagram of an embodiment in the present invention. As shown in FIG. 1, the head bone conduction device of the present embodiment includes a head fixing unit 19, an elongated contact arm 15 and a bone conduction unit 10. Referring to FIG. 1, the head fixing unit 19 includes a head support 13, a first clamping portion 11 and a second clamping portion 12.

As shown in FIG. 1, a head support 13, a first clamping portion 11 and a second clamping portion 12 forms a U-shaped head fixing unit 19, which would help the present invention be fixed to an appropriate position of a user's head with the head support 13 attached to an area of cranial cortex.

One end of the elongated contact arm 15 is connected to the bone conduction unit 10 while another end is connected to a first clamping portion 11 through a movable pivot 14. The elongated contact arm 15 can rotate around the movable pivot 14 and stretch to different lengths such that a bone conduction unit 10 will be able to attach to different areas of the cranial cortex of a user. After receipt of audio signals, the bone conduction unit 10 will trigger according mechanical vibration to the attached cranial cortex.

Referring to FIG. 1, a plurality of brain wave detection ends 17 is located on one side of the head support 13, to be specific, the inner side of the head support 13 which faces to cranial cortex. The brain wave detection end 17 is used for detecting the brain wave of a user. The detected brain wave from the brain wave detection end 17 can be transmitted to an external displaying device, such as an oscilloscope or computer, to display the brain waves. Moreover, the detected brain waves from the brain wave detection end 17 can be processed and the processed brain waves will be mixed with some received audio signals to change the mechanical vibration on cranial cortex including intensity, frequency or direction. As such, the user will sense a different audio signal after the mixing, which helps the user to identify his current learning status and fast-recalls this efficient learning status in future when needs. In addition, indicating light 16 can be located on another side of the head support 13 to indicate an operation status of the bone conduction 10.

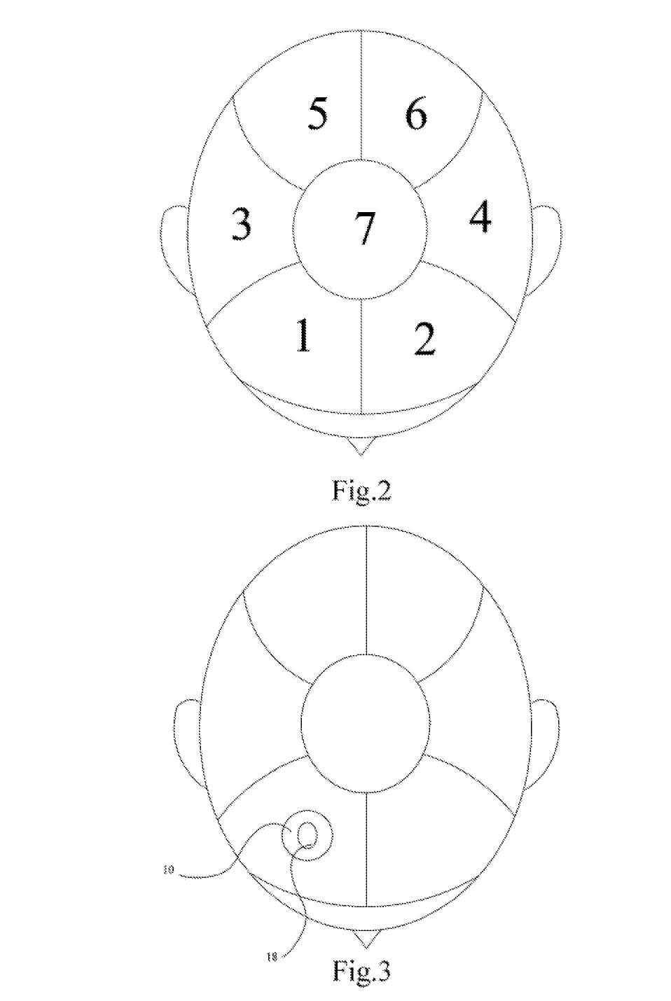

FIG. 2 is a partition view of a cranial cortex in an embodiment of the present invention. As shown in FIG. 2, the cranial cortex is divided into seven regions, including a region 1, a region 3 and a region 5 at the left side, a region 2, a region 4 and a region 6 at the right side, and a region 7 at the center. The bone conduction unit 10 can be attached to any one of the seven regions by rotating and stretching the elongated contact arm 15, so as to transmit the mechanical vibration to the according cranial cortex. Moreover, the position of the bone conduction unit 10 can be adjusted with different situations of cranial cortex to stimulate the user at a high efficiency. For example, due to the structural and functional difference between the left brain and the right brain, the bone conduction unit 10 can be attached on a region at the right brain while the brain wave detection end 17 is attached on a region at the left brain such that the right brain can act as a channel for inputting mechanical vibration while the left brain can act as a channel for brain wave feedback.

FIG. 3 is a schematic diagram of an embodiment of the present invention attached to a region of cranial cortex. Referring to FIG. 3, the bone conduction unit 10 further includes a phalanx conduction portion 18 which is located backward the cranial cortex of a user. When a user press down the phalanx conduction portion 18, such as by finger(s), the bone conduction unit 10 will get in touch with a region of cranial cortex and the phalanx conduction portion 18 will transmit the mechanical vibration to the user's finger. In other words, when a user press down the phalanx conduction portion 18, the bone conduction unit 10 will transmit the mechanical vibration to a region of cranial cortex and the finger which press down the phalanx conduction portion 18 at the same time.

When the bone conduction unit 10 receives an audio signal(s), the bone conduction unit 10 will convert the audio signal(s) into according mechanical vibration which will be transmitted to cranial cortex. There are three ways how the mechanical vibration acting on a cranial cortex. First, the mechanical vibration will stimulate the cerebral cortex through the auditory nerve network of inner ear(s), resulting in auditory stimulation on user which means the user will hear some sound. Meanwhile, the position where mechanical vibration acting on the cranial cortex will become the second channel for auditory stimulation on user which will also help the user to focus on where the mechanical vibration acts. This channel for auditory stimulation is critical for building up relationship between the received audio signal(s) with biological memory of a user. Further, when a user feels distracted or difficult to focus on learning, the phalanx conduction portion 18 will help a user to focus on the position where his finger feels the mechanical vibration. The mechanical vibration acting on his finger which presses down the phalanx conduction portion 18 will be transmitted from phalanx to skeleton to brain, forming the third channel for auditory stimulation which will help memorize. The activity level of the cerebral cortex would be enhanced due to the activation by mechanical vibration together with a high concentration of attention by self-control. Also, the blood supply to cerebral cortex is increased. As such, brain functions of the user would be much improved.

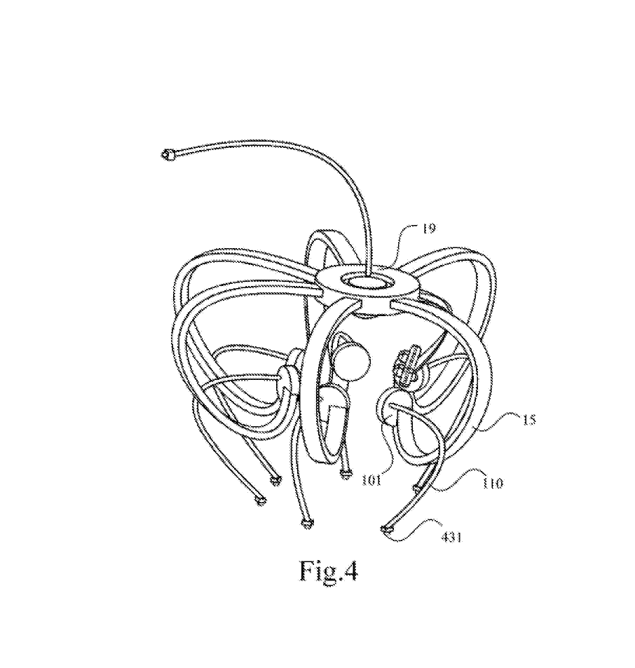

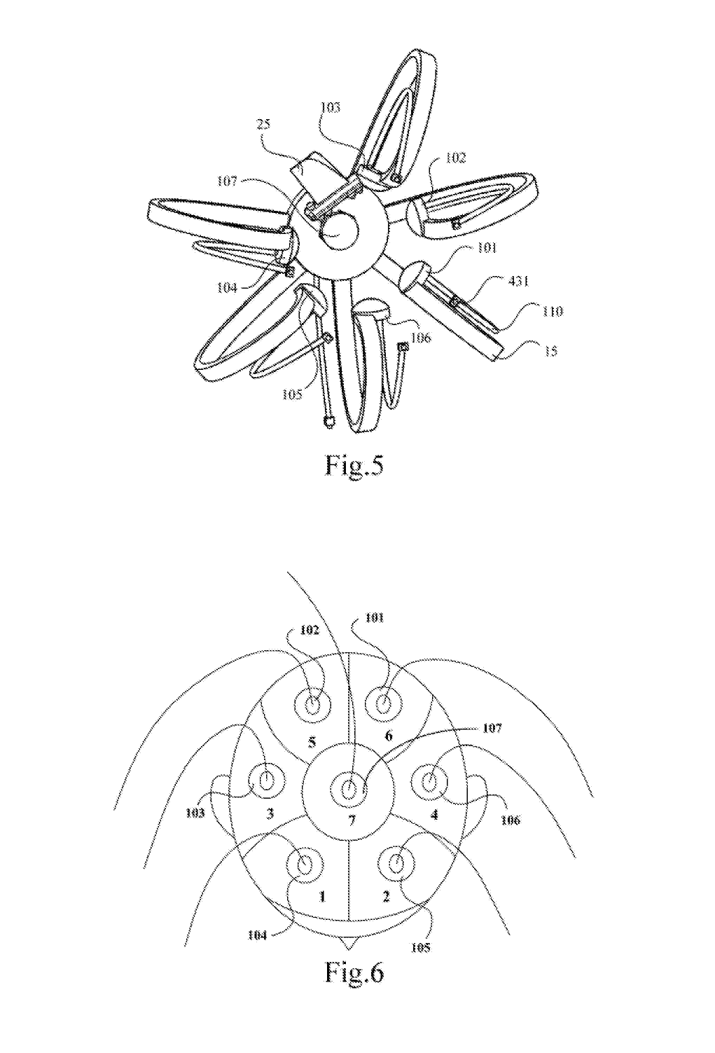

FIG. 4 is a structural schematic diagram of another embodiment of the present invention while FIG. 5 is a bottom view of FIG. 4. FIG. 6 is a schematic diagram of an application of the present invention with seven bone conduction units onto cranial cortex. Referring to FIG. 4-6, there are seven bone conduction units 10 in this embodiment, namely bone conduction units 101, 102, 103, 104, 105, 106 and 107. The seven bone conduction units connects with the head fixing units 19 through individual elongated contact arms 15 to form a helmet-like products. Seven bone conduction units 10 can be attached to seven individual regions of cranial cortex at the same time to transmit mechanical vibration on the whole cranial cortex to train the brain. Alternatively, the seven bone conduction units 10 can transmit the mechanical vibration in turn to stimulate different regions to find out the most active regions of cranial cortex.

In addition, referring to FIGS. 4 and 5, seven bone conduction units 101-107 connects individually with an elongated switch arm 110. The elongated switch arm is provided with a switch which is used for controlling the bone conduction units. Take the bone conduction unit 101 for example. The bone conduction unit 101 is connected with the head fixing unit 19 through the elongated contact arm 15. The switch 431 connects with the bone conduction unit 101 to control its operation. One end of the elongated switch arm connects with the bone conduction unit 101 while the other end connects with the switch 431. The elongated switch arm 101 can be made of flexible material and transmit the mechanical vibration from the bone conduction unit 101, which means the elongated switch arm 101 will vibrate in accordance with the bone conduction unit 101. As such, when a user touches the elongated switch arm 110 with his finger, he will feel the mechanical vibration from the bone conduction unit 101. By pressing the bone conduction unit 101 with finger(s), the bone conduction unit 101 will transmit mechanical vibration to phalanx. Meanwhile, the brain will react in response with the mechanical vibration based on physiological reflex. As such, similar as a set of exercise for muscle, a user will improve his brain functions by a combination of mechanical vibration on finger and concentrating.

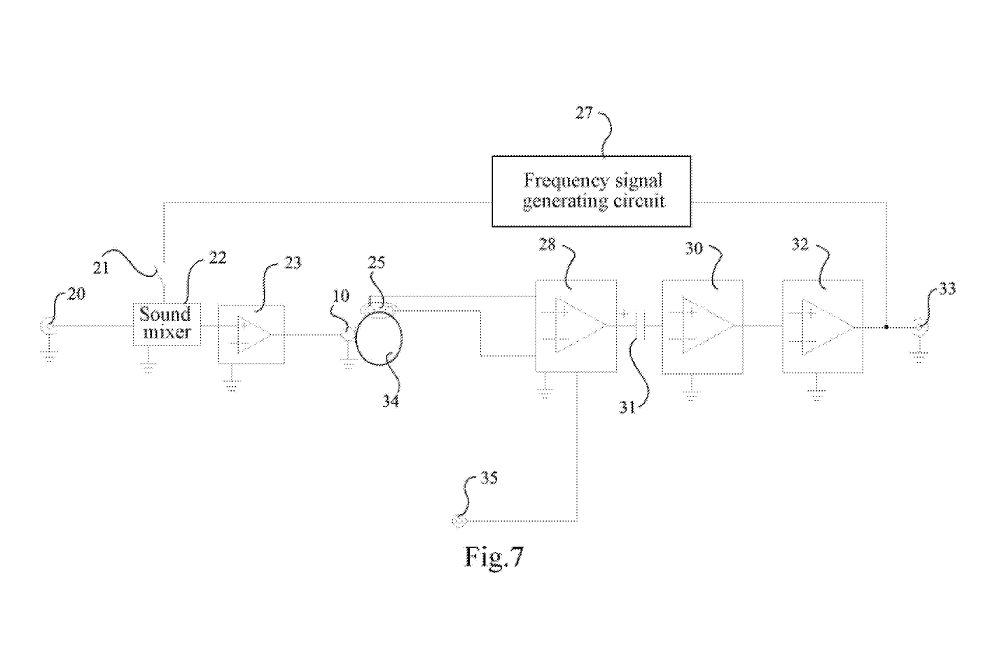

FIG. 7 is a circuit schematic of the present invention. As shown in FIG. 7, the circuit of the present invention mainly includes an audio transmitting part and a feedback part. The audio transmitting part includes an audio signal input end 20, an audio amplifier 23 and a bone conduction unit 10. The audio signal input end 20 is used for connecting with external audio input terminals, such as MP3 player, cell phone, computer and CD player, to receive audio signals(s). The audio amplifier 23 is used for amplifying the received audio signal(s) from audio signal input end 20 and transmitting the amplified audio signal(s) to the bone conduction unit 10.

As shown in FIG. 7, the feedback part of the circuit includes a brain wave detection end 25, a front stage amplifier 28, a frequency filter 31, a post stage amplifier 30, a low pass filter 32, a brain wave signal output end 33, a frequency signal generating circuit 27 and a sound mixer 22. The brain wave signal output end 33 is connected to a displaying device, such as an oscilloscope or monitor, to display the status of brain waves. The brain wave detection end 25, which is also known as the plurality of brain wave detection end 17 shown in FIG. 1, is connected to the front stage amplifier 28. The input end of the frequency filter 31 is connected to an output end of the front stage amplifier 28 while its output end is connected to an input end of the post stage amplifier 30. An input end of the low pass filter 32 is connected to an output end of the post stage amplifier 30 while its output end is connected to a brain wave signal output end 33. An electrode 35 serves as a public grounding loop for the brain wave detection end 25 which can be connected to human body. A sound mixer 22 is respectively connected to the audio signal input end 20, the frequency signal generating circuit 27 and the audio amplifier 23.

Referring to FIG. 7, the front stage amplifier 28 is used to amplify the brain wave signals and output the according amplified brain wave signals to the frequency filter 31 which filter the amplified brain wave signal from the front stage amplifier 28. The post stage amplifier 30 receives the filtered brain wave signal from the frequency filter 31 to compensate the attenuation and loss in amplitude of the electronic signals passing through the frequency filter 31. The low pass filter 32 is used to further filter the brain wave signals which comes from the post stage amplifier 30 to allow the specified brain wave signals to pass through, such as an α brain wave and/or a β brain wave. The brain wave signal output end 33 is connected to a displaying device, such as an oscilloscope or monitor, to display the brain waves for a user to observe the real time status. When alpha brain wave occurs which means the user being a super learning status, the user can grasp the opportunity to study efficiently.

A sound mixer 22 is used to mix the received audio signals, such as the audio signals from the audio signal input end 20 and the frequency signal generating circuit 27, and send the mixed audio signals to a bone conduction unit 10. By stimulating the brain of a user with the mixed audio signals, the brain will receive and be aware of a different mechanical vibration such that the brain will further confirm its current status is suitable for learning. The frequency signal generating circuit 27 generates different frequencies of signals within audible range based on the received brain wave signals which come from and have been filtered by the low pass filter 32.

Referring to FIG. 7, the operation process of a frequency signal generating circuit 27 and a sound mixer 22 will be described briefly. When the received brain waves are alpha brain wave with a spectrum between 7 Hz to 12 Hz, the frequency signal generating circuit 27 will correspondingly generates signals of some frequency signals, for example, a frequency signal of 600 Hz, and forward the frequency signal to a sound mixer 22. After the sound mixer 22 receives the frequency signals of 600 Hz, it mixes the frequency signals with the audio signals which come from the audio signal input end 20. The mixed signals then are forwarded to an audio amplifier 23 by which will be amplified to further forward to a bone conduction unit 10. As such, a bone conduction unit 10 will be able to output a different mechanical vibration which has been changed. The changed mechanical vibration, which has been adjusted based on the feedback from brain waves, enables a user to timely be aware of his brain status. Further, the changed mechanical vibration facilitates a user to focus on maintaining the output brain being alpha brave wave when he is distracted.

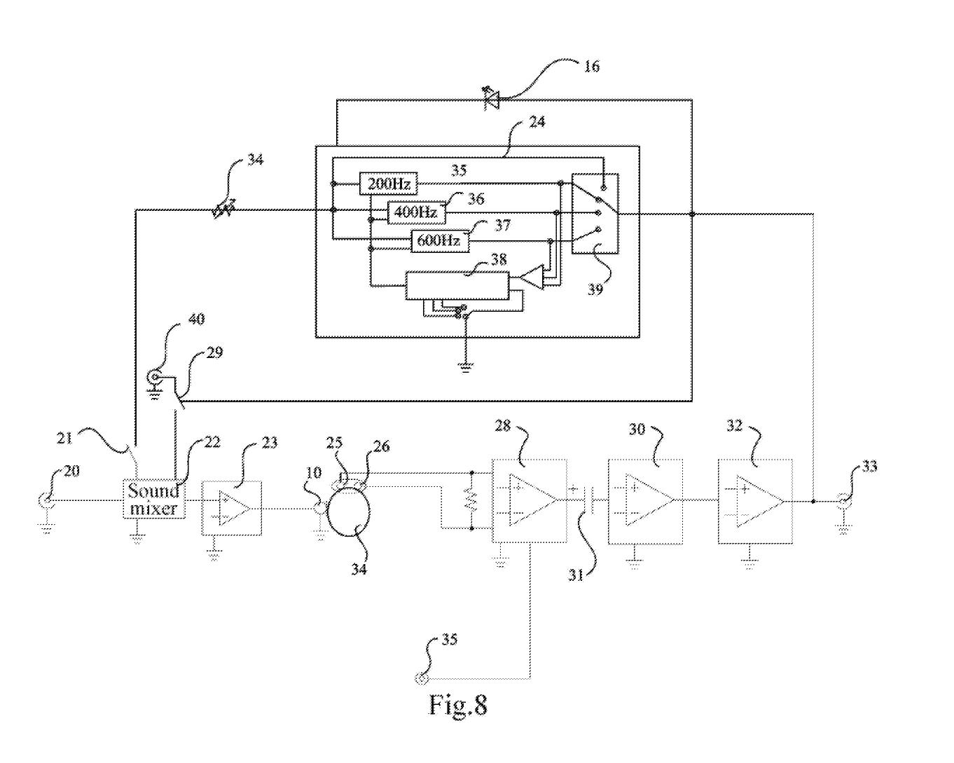

For a user who is unfamiliar to control his brain, he can induce the occurrence of alpha brain wave by the application of the audio signal input end 20 (shown in FIG. 8) and bone conduction units on the right brain. The combination of mechanical vibration and audio sense will not only induce the occurrence of alpha brain wave, but also help a user to recall such a high efficient status in future when he wants to.

Further referring to FIG. 7, a switch 21 is connected between a sound mixer 22 and a frequency signal generating circuit 27. As such, a user can select whether to mix the input audio signals or not to better experience the mechanical vibration by a bone conduction unit 10 when the brain is in the alpha brain wave state.

FIG. 8 is another schematic circuit diagram of a head bone conduction device of the present invention. As shown in FIG. 8, the frequency signal generating circuit 27 includes a frequency selector 39, a 200 Hz oscillator 35, a 400 Hz oscillator 36, a 600 Hz oscillator 37 and a timer 38.

In addition, referring to FIG. 8, the schematic circuit diagram further includes another audio input end 40, an indicating light 16 and a rheostat 34. As shown in FIG. 8, the rheostat 34 is used to adjust the magnitude of the frequency signals coming from a frequency signal generating circuit 27. The audio input end 40 is connected to an end of the low pass filter 32 and a sound mixer 22 through a switch 29. When the brain wave signals from the low pass filter 32 turn out as alpha brain wave signals, the switch 29 is closed to conduct the audio input end 40 and the sound mixer 22. Under this circumstance, different audio input modes may be adopted as follows:

1. When the brain wave signal from the low pass filter 32 is not an alpha brain wave signal, the audio signal input end 20 inputs a normal audio signal together with the frequency signal generating circuit 27, to induce alpha brain waves.

2. When the brain wave signal from the low pass filter 32 is an alpha brain wave signal, the switch 29 is triggered to be closed, and the audio input end 40 and the audio signal input end 20 are allowed to input a same audio signal.

3. When the brain wave signal from the low pass filter 32 is an alpha brain wave signal, the switch 29 is triggered to be closed, and the audio input end 40 and the audio signal input end 20 are allowed to input different audio signals.

The switch 29 may be triggered by an alpha brain wave. That is, an audio signal is input from an audio signal input end 20 by a user and some bone conduction unit(s) 10 is placed on the scalp of the right brain. The combination of mechanical vibration and audio sense would induce the occurrence of alpha brain waves. After a user become familiar with such operation process, a user will be able to induce the occurrence of alpha brain wave by oneself based on his previous memory. When the alpha brain waves occur, the switch 29 of the audio input end 40 can be triggered to be switched on, such that a user can hear an audio signal which comes from the audio input end 40.

However, operation is not limited to the above few ways. A user can adopt different operation methods with the audio input end 40 and the audio signal input end 20 according to different situation.

A frequency selector 39 is used to conduct different oscillators depending on different frequency of the input original brain wave signal. For example, the 200 Hz oscillator 35 is used to trigger a 200 Hz frequency signal, the 400 Hz oscillator 36 is used to trigger a 400 Hz frequency signal and the 600 Hz oscillator 37 is used to trigger a 600 Hz frequency signal. The indicating light 16 is triggered on upon detection of a brain wave with spectrum within 7-12 Hz (alpha brain wave). Moreover, in the present embodiment, the original brain wave signal is an alpha brain wave. In fact, the frequency ranges of the oscillators are not limited to the above. Any audible frequency between 20 Hz and 200 Hz of the oscillators can be adopted. However, as low frequencies of 200 Hz, 400 Hz or 600 Hz presents a better audio sense, oscillators of 200 Hz, 400 Hz and 600 Hz are preferable in the present invention.

In addition, when the audio signal input end 20 is not working or unloaded, that is, there is no signal input to the audio signal input end 20, the sound mixer 22 will directly outputs the frequency signal from the frequency signal generating circuit 27.

When the brain wave signal from the low pass filter 32 is an alpha brain wave signal, the frequency selector 39 triggers a 200 Hz signal from the 200 Hz oscillator 35. When the frequency of brain wave signal from the low pass filter 32 is between 7-9 Hz, the frequency selector 39 triggers a 400 Hz signal from the 400 Hz oscillator 36. When the frequency of brain wave signal from the low pass filter 32 is between 10-12 Hz, the frequency selector 39 triggers a 600 Hz signal from the 600 Hz oscillator 37. Alternatively, the brain wave signals of a user can be feedback into the present invention via a line 24 which loads no frequency signal. To be specific, a frequency selector 39 would identify the brain wave signal from the low pass filter 32 whether it is an alpha brain wave signal. The identified alpha brain wave would be further classified, namely (7 Hz-9 Hz), (10 Hz-12 Hz) and (7 Hz-12 Hz). As such, the frequency selector 39 would be able to start four different independent frequency signals, namely 200 Hz, 400 Hz or 600 Hz frequency signal and so on, as well as controlling cycles.

Each of the four different independent frequency signals can be forwarded to a bone conduction unit 10 via a sound mixer 22 and an audio amplifier 23. Further, as the detected alpha brain wave may be discontinuous, a timer 38 is set to a specified period, such as 0.5 second, 1 second or 1.5 second to delay the triggered frequency signal(s) from an oscillator to according duration, namely 0.5 second, 1 second or 1.5 second. As such, a user can identify the alpha brain wave easily even the detected alpha brain wave is discontinuous. For a user who has been able to easily identify the alpha brain wave, a timer 38 can be set as 0 second which means no delay for the triggered frequency signal(s). A user can manually select and switch the functions of both timer 38 and frequency selector 39.

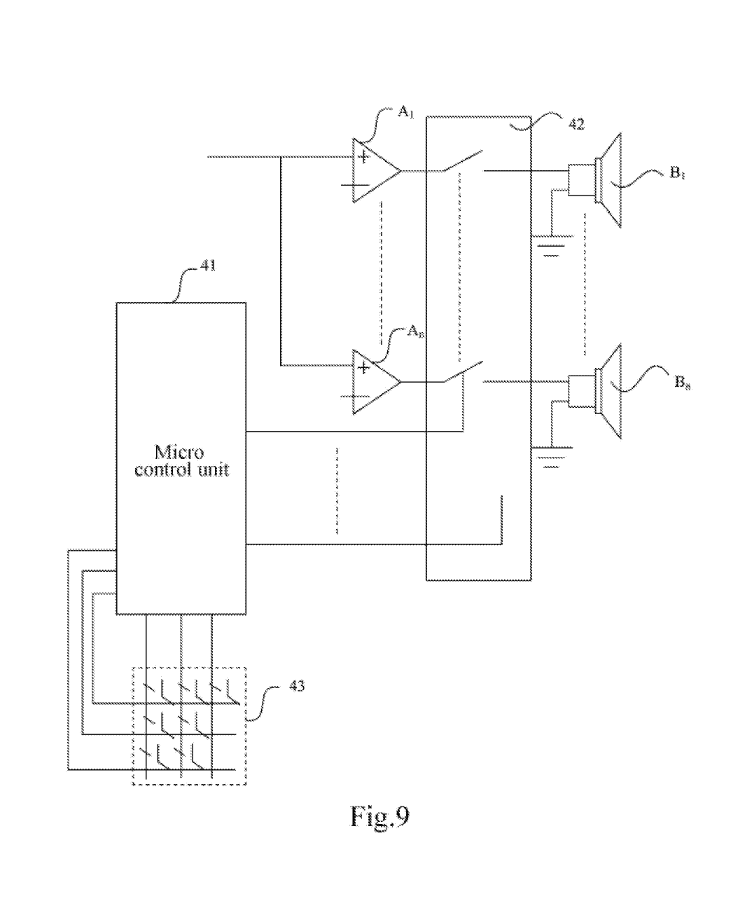

FIG. 9 is a schematic diagram of a control circuit of the present invention having seven bone conduction units. Referring to FIG. 9 in conjunction with FIG. 8, in this embodiment, there are seven bone conduction units and seven audio amplifiers, namely bone conduction units B₁˜Bₙ and audio amplifiers A₁˜Aₙ. Each bone conduction unit corresponds to a single audio amplifier. Also, all seven audio amplifiers are connected with the sound mixer 22. A set of switches 42 is connected between the bone conduction units B₁˜Bₙ and the audio amplifiers A₁˜Aₙ to control the on-off between the bone conduction units B₁˜Bₙ and the audio amplifiers A₁˜Aₙ. In FIG. 8, another set of switches 43 is used to control the on-off of each bone conduction units B₁˜Bₙ. A user can select one or more bone conduction unit(s) to work by selecting the according number of switch(es) 43. A micro control unit 41 sends the according switch signal to the set of switches 42 according to the user's selection of the set of switches 43, to control which bone conduction unit(s) B₁˜Bₙ to be turned on for operation. Take the switch 431 in FIG. 4 for example, in which the switch 431 is one switch of the set of switches 43. By manually turn on or off the switch 431, the bone conduction unit 101 can be turned on or off accordingly. Also, the on-off of the seven conduction units can be programmed and executed by the micro control unit 41, which enables the seven bone conduction units conduct mechanical vibration in some sequence, in random individually, or in different combination. Further, it is feasible to operate the seven conduction units by program and/or manually.

Referring to FIGS. 1 to 9, a head bone conduction method according to an embodiment of the present disclosure includes:

attaching at least one bone conduction unit to a region of a cranial cortex to conduct mechanical vibration;

detecting a brain wave signal from cranial cortex by a brain wave detection end;

generating at least one frequency signal based on the detected brain wave signal; and

transforming a mixed signal into mechanical vibration, wherein the mixed signal comes from a first audio signal mixed with the frequency signal.

Regarding the steps of detecting a brain wave signal from cranial cortex and generating at least one frequency signal based on the detected brain wave signal, it would further includes the steps as follows:

detecting a brain wave signal from cranial cortex;

judging whether the detected brain wave signal is an alpha brain wave or not;

if the detected brain wave signal is an alpha brain wave signal, transforming a mixed signal into mechanical vibration wherein the mixed signal comes from a first audio signal mixed with the alpha brain wave signal.

Regarding the step of generating at least one frequency signal within audible range based on the brain wave signal, it would further includes the steps as follows:

judging whether the detected brain wave signal is an alpha brain wave or not;

if the detected brain wave signal is an alpha brain wave signal, classifying the detected brain wave according to different frequency; and

generating the frequency signal based on the detected brain wave.

In addition, a triggering time for the frequency signal can be adjusted by a timer.

In another embodiments, the head bone conduction method can further include the following steps:

Dividing the cranial cortex into seven regions, wherein three regions are located in the left side, another three regions are located in the right side and a region is located at the center;

When the brain wave signal is an alpha brain wave, a bone conduction unit will get attached to different regions to identify which region the alpha brain wave comes from.

Alternatively, the head bone conduction method can further include: seven bone conduction units get attached to seven regions individually to conduct mechanical vibration to these seven regions in random or in sequence.

Further, the head bone conduction method includes: detecting the brain wave from cranial cortex to monitor the status of brain wave.

In a third embodiment, the head bone conduction method can further include the following steps:

Pressing the bone conduction unit by finger to conduct mechanical vibration from the bone conduction unit to phalanx, such that a user will be able to identify the specific position where a combination of mechanical vibration sense from cranial cortex and from finger acting on.

In other embodiment, the head bone conduction method can further include:

Transmitting a processed brain wave signal to a brain wave displaying unit, wherein the processed brain wave signal is the detected brain wave which has been amplified and filtered;

Meanwhile, synthesizing the processed brain wave signal into a first audio signal, and the first audio signal will be mixed with the audio signal from the head bone conduction device;

Amplifying the mixed brain wave signal; and

Forwarding the amplified brain wave signal to at least one bone conduct unit(s).

Moreover, the head bone conduction method can further include: when the brain wave signal is an alpha brain wave, inputting a second audio signal and mixing it with at least one frequency signal.

In addition, different operation methods shall be adopted according to users who have different abilities in concentration. Take a user who is poor in controlling brain functioning for example. It would be preferable to activate a plurality of bone conduction units to enable the user experience how the brain works with different regions stimulated by the bone conduction units. Take a user who has some basic knowledge in controlling brain functioning for another example. When the user is concentrated, alpha brain wave would be easily detected. Meanwhile, a plurality of bone conduction units can also be activated on the user to help user focus. For a user who are familiar with controlling brain functioning, only the detecting units would be needed to confirm the occurrence of alpha brain wave and it would be no need to further trigger alpha brain wave.

For a user with insufficient amount of brain activity, it would benefit the user by the combined audio sense and vibration sense which would strength brains' functioning. A further combination of brain wave detecting would be much advantageous to help a user change his brain wave.

As such, in the present disclosure, bone conduction units are individually attached to the cranial cortex to conduct mechanical vibration. Meantime, attention is drawn to the region where mechanical vibration acting on to awake the cranial cortex. After such a period of action, the brain would turn to a status of receiving mechanical vibration. By combining the brain wave signal from the cranial cortex and audio signal from an external audio device, a user would judge his own status when he realizes the change of mechanical vibration. Further, attention is kept drawn to the regions where mechanical vibration acting on the cranial cortex such that the attention would become long-lasting, which means enhancing mental focus. Moreover, the present invention helps the brain functioning and prevents brain problems such as memory loss. Further, comparing with the existing head bone conduction device, the present invention is simpler in structure and not susceptible to interference from other components such as headphones.

The present invention has been described in detail, with reference to the preferred embodiment, in order to enable the reader to practice the invention without undue experimentation. However, a person having ordinary skill in the art will readily recognize that many of the previous disclosures may be varied or modified somewhat without departing from the spirit and scope of the invention. Accordingly, the intellectual property rights to this invention are defined only by the following claims.

What is claimed is:

1. A head bone conduction device, comprising:
a sound mixer which is configured to receive at least one frequency signal within audible frequency, and mix said frequency signal with a first audio signal to obtain audio information;
at least one bone conduction unit which is configured to convert said audio information from the sound mixer into mechanical vibration, and conduct the mechanical vibration to a region of cranial cortex which is attached to;
at least one brain wave detection end which is configured to detect brain wave signal of the cranial cortex; and
a frequency signal generating circuit which is configured to generate the frequency signal within audible frequency based on the detected brain wave signal from the brain wave detection end.

2. The head bone conduction device according to claim 1, wherein the frequency signal generating circuit further comprises:
a plurality of oscillators which are connected in parallel to generate frequency signals within audible frequency;
a frequency selector which is connected to the plurality of oscillators in series to conduct one of the oscillators when the detected brain wave signal is an alpha brain wave; and
a timer which is connected to the plurality of oscillators in series and configured to control a delaying period to conduct the oscillators.

3. The head bone conduction device according to claim 2, wherein the plurality of oscillators further comprises a 200 Hz oscillator, a 400 Hz oscillator and a 600 Hz oscillator; and
the delaying period by the timer is 0 second, 0.5 second, 1 second or 1.5 seconds.

4. The head bone conduction device according to claim 2, wherein the device further comprises:
a front stage amplifier which is connected to a brain wave detection end to amplify the brain wave signal detected by the brain wave detection end for a first time;
a frequency filter which is connected to the front stage amplifier to filter the amplified brain wave signal from the front stage amplifier;
a post stage amplifier which is connected to the frequency filter to amplify the filtered brain wave signal from the frequency filter for a second time and compensate attenuation and loss in amplitude; and
a low pass filter which is connected to the post stage amplifier to receive the second amplified brain wave signal from the post stage amplifier and to allow a brain wave signal of specific frequency to pass through; and
a brain wave signal output end which is connected to the low pass filter to receive the brain wave signal filtered by the low pass filter.

5. The head bone conduction device according to claim 1, wherein the device further comprises an indicating light to be triggered when the detected brain wave signal from the brain wave detection end is alpha brain wave.

6. The head bone conduction device according to claim 1, wherein the frequency signal generating circuit is further configured to feedback the detected brain wave signal from the brain wave detection end to the sound mixer; and
the sound mixer mix the detected brain wave signal with a first audio signal to obtain an audio information, and send the audio information to at least one bone conduction unit.

7. The head bone conduction device according to claim 1, wherein the device further comprises a manual switch which is connected between the frequency signal generating circuit and the sound mixer.

8. The head bone conduction device according to claim 1, wherein the device further comprises at least one audio amplifier whose quantity is in correspondence with the number of the bone conduction unit, and said audio amplifier is connected between the sound mixer and the bone conduction unit.

9. The head bone conduction device according to claim 1, wherein the device further comprises a first audio apparatus to provide a first audio signal.

10. The head bone conduction device according to claim 9, wherein the device further comprises a second audio apparatus to provide a second audio signal to the sound mixer when the brain wave signal detected by the brain wave detection end is an alpha brain wave.

11. The head bone conduction device according to claim 1, wherein the device comprises more than one bone conduction units and each bone conduction units conducts its mechanical vibration in sequence or in random.

12. The head bone conduction device according to claim 1, wherein the bone conduction unit comprises a phalanx conduction portion which is located backward the cranial cortex to conduct the mechanical vibration from the bone conduction unit to a phalanx.

13. The head bone conduction device according to claim 1, wherein the brain wave signal is an alpha brain wave signal.

14. The head bone conduction device according to claim 1, wherein the device further comprises:
a head fixing unit to support and secure the device to the head of a user, the head fixing unit is of a “U” shaped frame structure and comprises a head support at an upper portion and two opposite clamping portions at side portions;
at least one brain wave detection end is located inner side of the head support of the head fixing unit;
at least one elongated contact arm, one end of each elongated contact arm movably is connected to the head fixing unit while another end connected to the bone conduction unit.

15. The head bone conduction device according to claim 1, wherein the device further comprises a head fixing unit to support and secure the device to the head of a user, the head fixing unit comprises:
a head support;
a plurality of elongated contact arms which are located around the head support, one end of each elongated contact arms is connected to the head fixing unit, while another end of each elongated contact arms is connected to the brain wave detection end and/or the bone conduction unit;
and, the plurality of elongated contact arms can be clamped to the head of a user to attach the one brain wave detection end and the bone conduction unit to a scalp of the cranial cortex.

16. The head bone conduction device according to claim 15, wherein the device comprises seven bone conduction units, and the seven bone conduction units are connected to the head support respectively via seven elongated contact arms to form a helmet shape.

17. The head bone conduction device according to claim 1, wherein the device further comprises at least one switch, each switch is correspondingly connected to the bone conduction unit.

18. The head bone conduction device according to claim 17, wherein the device further comprises at least one elongated switch arm which is connected to the corresponding bone conduction unit to conduct mechanical vibration, each elongated switch arm is provided with an individual switch to control the corresponding bone conduction unit.

19. The head bone conduction device according to claim 17, wherein the device further comprises a micro unit which is connected to the switch to control on-off between the sound mixer and the bone conduction unit according to the switch of the elongated switch arm.

20. A head bone conduction method, comprising:
attaching at least one bone conduction unit to a region of a cranial cortex to conduct mechanical vibration;
detecting a brain wave signal of the cranial cortex by the brain wave detection end;
generating at least one frequency signal based on the brain wave signal detected by the brain wave detection end; and
transforming a mixed signal into mechanical vibration, wherein the mixed signal comes from a first audio signal mixed with the frequency signal.

21. The head bone conduction method according to claim 20, wherein generating at least one frequency signal based on the brain wave signal detected by the brain wave detection end further comprises:
judging whether the detected brain wave signal is an alpha brain wave or not;
if the detected brain wave signal is an alpha brain wave signal, classifying the detected brain wave signal according to different frequency; and
generating the frequency signal based on the detected brain wave signal.

22. The head bone conduction method according to claim 21, wherein the method further comprises: adjusting a triggering time for the frequency signal by a timer.

23. The head bone conduction method according to claim 20, wherein the method further comprises:
dividing the cranial cortex into seven regions, wherein three regions are located in the left side, another three regions are located in the right side and a region is located at the center;
when the brain wave signal is an alpha brain wave, a bone conduction unit gets attached to different regions to identify which region the alpha brain wave comes from.

24. The head bone conduction method according to claim 20, wherein seven bone conduction units individually get attached to seven regions of cranial cortex to conduct mechanical vibration in random or in sequence.

25. The head bone conduction method according to claim 20, wherein detecting a brain wave signal of the cranial cortex by the brain wave detection end and generating at least one frequency signal based on the brain wave signal detected by the brain wave detection end, further comprises:
judging whether the detected brain wave signal is an α brain wave or not,
if the detected brain wave signal is an alpha brain wave signal, transforming a mixed signal into mechanical vibration wherein the mixed signal comes from a first audio signal mixed with the alpha brain wave signal.

26. The head bone conduction method according to claim 20, wherein the method further comprises:
pressing the bone conduction unit by finger to conduct mechanical vibration from the bone conduction unit to phalanx to enable a user to identify the specific position where a combination of mechanical vibration sense from cranial cortex and from finger acting on.

27. The head bone conduction method according to claim 20, wherein the method further comprises: if the detected brain wave signal is an alpha brain wave signal, a second audio signal is inputted from the second audio apparatus and be mixed with the frequency signal.
